(19) United States
(12) Patent Application Publication
(10) Pub. No.: US 2025/0237587 A1
(43) Pub. Date: Jul. 24, 2025

(54) METHOD AND SYSTEM FOR CHARACTERIZING RHEOLOGICAL CHARACTERISTICS OF SUBMARINE LANDSLIDE MASS

(71) Applicant: SHANDONG UNIVERSITY, Jinan, Shandong (CN)

(72) Inventors: Shucai LI, Jinan (CN); Zongqing ZHOU, Jinan (CN); Liping LI, Jinan (CN); Yuxue CHEN, Jinan (CN); Hongliang LIU, Jinan (CN); Shuai CHENG, Jinan (CN); Cong LIU, Jinan (CN); Jing WANG, Jinan (CN); Shang GAO, Jinan (CN)

(73) Assignee: SHANDONG UNIVERSITY, Jinan, Shandong (CN)

(21) Appl. No.: 18/832,447

(22) PCT Filed: May 23, 2023
(86) PCT No.: PCT/CN2023/095863

§ 371 Date: Jul. 23, 2024

(30) Foreign Application Priority Data
Oct. 27, 2022 (CN) 202211324763.2

(51) Int. Cl. G01N 3/24; G01N 11/00; G01N 33/18; G01N 33/24

(52) U.S. Cl. CPC G01N 3/24; G01N 11/00; G01N 33/18; G01N 33/24; G01N 2011/0033; G01N 2203/0025; G01N 2203/0218

(57) ABSTRACT

A method for characterizing thixotropic characteristics of submarine landslide mass material, including: performing steady shear on landslide mass material, and determining solid-fluid transition boundary according to variation of yield stress value in yield stress test; performing conversions between shear speed and shear rate and between shear torque and shear stress according to variations of apparent viscosity-shear speed and shear torque-shear speed of landslide mass material at different shear times in apparent viscosity test to obtain variations of the apparent viscosity and the shear stress at different shear rates to construct conventional rheological model; constructing thixotropy characterization equation according to variations of the shear stress and the apparent viscosity with the shear time in the thixotropy test; and constructing rheological model for characterizing the thixotropic characteristics of the submarine landslide mass material according to the solid-fluid transition boundary, the conventional rheological model, and the thixotropy characterization equation.

The present disclosure claims priority to Chinese Patent Application No. 202211324763.2 filed with the National Intellectual Property Administration on Oct. 27, 2022 and entitled “METHOD AND SYSTEM FOR CHARACTERIZING THIXOTROPIC CHARACTERISTICS OF SUBMARINE LANDSLIDE MASS MATERIAL”. The entire content of the above-referenced disclosure is incorporated herein by reference.

### TECHNICAL FIELD

The present disclosure relates to the technical field of marine engineering, and in particular, to a method and a system for characterizing thixotropic characteristics of a submarine landslide mass material.

### BACKGROUND

Statements in this part only provide background technical information related to the present disclosure and do not necessarily constitute a prior art.

Submarine landslides are common geological disasters in marine environment. Except for the landslides caused by submarine earthquakes, deep-sea volcanic eruptions, and the like, the submarine landslides often occur at an edge of an offshore continental shelf. During a submarine landslide, especially, before and after an offshore silt seabed landslide, there will be problems about flowing and transporting of high-concentration viscous sediment. A motion law of a large amount of sediment is affected by multiple factors, such as rheological characteristics and transportation movement. However, the rheological characteristics of the viscous sediment are also an important factor that affects the transportation movement of the viscous sediment.

Rheological phenomena of the viscous sediment widely exist in offshore engineering under an action of a shear load. With frequent occurrences of extreme weather conditions, hidden dangers of offshore submarine landslides are gradually increasing. In addition, concealment of submarine landslide disasters and immature monitoring and early warning technologies for the submarine landslide disasters pose a serious threat to safety production of coastal and submarine engineering sites and underwater structures in an offshore area.

An offshore silt seabed landslide mass may be considered as a high-concentration viscous mud-sand-water mixture. Rheological characteristics of this mixture and a similar dispersion system may vary with a shear time. This complex thixotropic phenomenon cannot be ignored. However, at a present stage, sediment beds are mainly researched based on a sediment theory. When sand content of a water body exceeds a limit, a constitutive relationship (namely, a relationship between a stress and a strain rate) of the sediment mainly adopts pure viscosity non-Newtonian fluid rheological models such as a Bingham body, pseudoplastic body, a dilatant model, and a two-phase flow model. However, the above rheological models do not consider an impact of shear duration. The rheological models are independent of time. The shear stress is only a function of a corresponding shear rate.

In addition, research has found that high-concentration sediment has a phenomenon of solid-liquid phase transition under an action of shearing. This phase transition is crucial for mud-sand stratification characteristics and mutual transition between layers. Due to complex compositions and structures of seabed sediment, especially, in the presence of an amount of fine particles, a relationship between the shear stress and the shear rate of the seabed sediment usually varies with the shear time and shear history of the seabed sediment. In such cases, the mud-sand-water mixture is to be considered as a time dependent non-Newtonian fluid. Therefore, research on rheological characteristics of the offshore silt seabed sediment is urgently to be carried out.

The rheological model is to establish a relationship between stress and deformation, while theoretical derivation is usually only applicable to a simplified ideal calculation situation and requires sufficient experimental verification. A numerical simulation method for a complex rheological/flow behavior only provides qualitative explanations for a complex rheological behavior of part typical yield stress fluids, but cannot quantitatively characterize the complex rheological behavior of a mixed system of a landslide mass, and still requires a large amount of summary work to systematically understand causes of the complex rheological behavior.

In conclusion, a conventional rheological model is only constructed through an apparent viscosity-shear rate relationship or a shear stress-shear rate relationship in existing research, resulting in that it is difficult for the model to reflect an impact of thixotropy on apparent viscosity. Or, separate research on the thixotropy of a material system cannot effectively determine a solid-fluid boundary of the material system, which results in that a thixotropy test/characterization time is too long, duration is unpredictable, and is difficult to timely and effectively characterize the thixotropy of the material system.

### SUMMARY

To solve the problems above, the present disclosure provides a method and a system for characterizing thixotropic characteristics of a submarine landslide mass material. A rheological model capable of characterizing the thixotropic characteristics of the submarine landslide mass material is constructed to clarify steady-state rheological characteristics and flow behaviors of the submarine landslide mass material, so as to provide effective theoretical guidance for researching transportation and accumulation of a landslide mass during a submarine landslide disaster.

To achieve the above objectives, the present disclosure adopts the following technical solutions:

According to a first aspect, the present disclosure provides a method for characterizing thixotropic characteristics of a submarine landslide mass material, including:
performing steady shear on a landslide mass material by setting a shear speed and a shear time to perform a yield stress test, an apparent viscosity test, and a thixotropy test on the landslide mass material;
determining a solid-fluid transition boundary of the landslide mass material according to a varying law of a yield stress value with the shear time in the yield stress test;
performing conversions between a shear speed and a shear rate and between a shear torque and a shear stress according to varying laws of apparent viscosity-shear speed and shear torque-shear speed of the landslide mass material at different shear times in the apparent viscosity test to obtain varying laws of the apparent viscosity and the shear stress at different shear rates to construct a conventional rheological model of the landslide mass material;
constructing a thixotropy characterization equation of the landslide mass material according to the varying laws of the shear stress and the apparent viscosity with the shear time in the thixotropy test; and
constructing a rheological model for characterizing the thixotropic characteristics of the submarine landslide mass material according to the solid-fluid transition boundary, the conventional rheological model, and the thixotropy characterization equation to obtain steady-state rheological characteristics and flow behaviors of the submarine landslide mass material.

As an optional embodiment, in the yield stress test, a constant shear speed is set to perform steady shear yield stress tests at different moisture contents to obtain a yield characteristic curve of the landslide mass material. After a certain shear duration, whether the landslide mass material tends to a steady shear stress value is determined according to a variation of the shear stress value of the landslide mass material with the shear time, and the yield stress value at this moment is a critical yield stress of the solid-fluid transition boundary after the landslide mass material yields.

As an optional embodiment, in the thixotropy test, the thixotropy test at a corresponding shear speed and shear time is performed based on different shear speeds and shear times in the apparent viscosity test to obtain variations of the shear stress and the apparent viscosity with the shear time.

As an optional embodiment, the thixotropy test at the corresponding shear speed and shear time is as follows: the shear speed in the apparent viscosity test gradually increases to a maximum shear speed, the same shear time is maintained after the shear speed reaches a maximum value in the thixotropy test, the shear speed is decreased sequentially corresponding to a shear speed increasing process and the corresponding shear time in the apparent viscosity test, and the corresponding shear time is maintained.

As an optional embodiment, a time dependent thixotropy characterization coefficient is introduced in the thixotropy test to quantitatively characterize non-Newtonian fluid characteristics of the landslide mass material.

As an optional embodiment, the time dependent thixotropy characterization coefficient K is as follows:

$$K = \frac{1}{n} {\sum }_{i = 1}^{n} ( \frac{{η}_{1} - {η}_{i}}{In ( {t}_{i} - {t}_{1} ) In ( {\dot{γ}}_{i} - {\dot{γ}}_{1} )} )$$

wherein, i is an i^{th} duration of the shear rate, n is an entire shear duration, [symbol] and [symbol] are the shear rates corresponding to time t₁ and time tᵢ, and η₁ and ηᵢ are apparent viscosities of the system corresponding to time t₁ and time tᵢ.

As an optional embodiment, a landslide mass material sample is prepared by comprehensively considering particle size gradation and moisture content of landslide mass material components; or, sample remodeling is performed by using samples collected on site to obtain landslide mass material samples with different moisture contents.

According to a second aspect, the present disclosure provides a system for characterizing thixotropic characteristics of a submarine landslide mass material, including:
a test module, configured to perform steady shear on a landslide mass material by setting a shear speed and a shear time to perform a yield stress test, an apparent viscosity test, and a thixotropy test on the landslide mass material;
a first response relationship obtaining module, configured to determine a solid-fluid transition boundary of the landslide mass material according to a varying law of a yield stress value with the shear time in the yield stress test;
a second response relationship obtaining module, configured to perform conversions between a shear speed and a shear rate and between a shear torque and a shear stress according to varying laws of apparent viscosity-shear speed and shear torque-shear speed of the landslide mass material at different shear times in the apparent viscosity test to obtain varying laws of the apparent viscosity and the shear stress at different shear rates to construct a conventional rheological model of the landslide mass material;
a third response relationship obtaining module, configured to construct a thixotropy characterization equation of the landslide mass material according to the varying laws of the shear stress and the apparent viscosity with the shear time in the thixotropy test; and
a thixotropic characteristic characterization module, configured to construct a rheological model for characterizing the thixotropic characteristics of the submarine landslide mass material according to the solid-fluid transition boundary, the conventional rheological model, and the thixotropy characterization equation to obtain steady-state rheological characteristics and flow behaviors of the submarine landslide mass material.

According to a third aspect, the present disclosure provides an electronic device, including a memory, a processor, and computer instructions stored in the memory and running in the processor. The method according to the first aspect is completed when the computer instructions are run by the processor.

According to a fourth aspect, the present disclosure provides a computer-readable storage medium, configured to store computer instructions. The method according to the first aspect is completed when the computer instructions are executed by a processor.

Compared with a related art, the present disclosure has the following beneficial effects:

The present disclosure provides a method and a system for characterizing thixotropic characteristics of a submarine landslide mass material. A rheological model for characterizing the thixotropic characteristics of the submarine landslide mass material is constructed, solid like deformation and liquid like flow characteristics of a non-Newtonian fluid are comprehensively considered, a solid-fluid boundary of a fluid mixed system is determined, and a conventional rheological model and a thixotropy characterization equation of a fluid are constructed. An impact of a shear time on non-Newtonian fluid characteristics of the fluid mixed system is further quantitatively characterized based on quantifying a traditional shear stress-shear rate relationship, a rheological behavior in an offshore silt seabed landslide mass/sediment transporting and sliding process is clarified, and effective theoretical guidance is provided for researching transportation and accumulation of a landslide mass during a submarine landslide disaster.

Advantages of additional aspects in the present disclosure will be partially given in the following description, and some will become apparent from the following description, or will be understood by the practice of the present disclosure.

### BRIEF DESCRIPTION OF THE DRAWINGS

Accompanying drawings constituting a part of the present disclosure are used for providing a further understanding of the present disclosure. Illustrative embodiments of the present disclosure and descriptions thereof are used for explaining the present disclosure, and do not constitute an improper limitation to the present disclosure.

FIG. 1 is a flowchart of a method for characterizing thixotropic characteristics of a submarine landslide mass material according to Embodiment 1 of the present disclosure;

FIG. 2(a) is a schematic diagram of a varying curve of a shear torque and a shear time according to Embodiment 1 of the present disclosure;

FIG. 2(b) is a schematic diagram of a stress-strain curve according to Embodiment 1 of the present disclosure;

FIG. 3(a) is a schematic diagram of an apparent viscosity-shear speed rheological according to Embodiment 1 of the present disclosure;

FIG. 3(b) is a schematic diagram of a shear torque-shear speed rheological curve according to Embodiment 1 of the present disclosure;

FIG. 4(a) is a schematic diagram of an apparent viscosity-shear speed-shear time response relationship according to Embodiment 1 of the present disclosure; and

FIG. 4(b) is a schematic diagram of a shear torque-shear speed-shear time response relationship according to Embodiment 1 of the present disclosure.

### DETAILED DESCRIPTION

The present disclosure is further described below with reference to drawings and embodiments.

It is to be noted that the following detailed descriptions are all exemplary, and are intended to provide further descriptions of the present disclosure. Unless otherwise specified, all technical and scientific terms used herein have the same meanings as those usually understood by those of ordinary skill in the art to which the present disclosure belongs.

It is to be noted that terms used herein are merely used for describing specific embodiments, and are not intended to limit exemplary embodiments of the present disclosure. As used here, unless otherwise specified in the context, a singular form is alternatively intended to include a plural form. In addition, it is also to be understood that terms “include” and “have” and any other variants thereof are intended to cover non-exclusive inclusion, for example, processes, methods, systems, products, or devices that include a series of operations or units are not necessarily limited to those clearly listed operations or units, but may include other operations or units not clearly listed or inherent to these processes, methods, systems, products, or devices.

The embodiments in the present disclosure and features in the embodiments may be combined with each other without conflict.

### Embodiment 1

In response to an existing conventional rheological model not fully considering non-Newtonian fluid flow characteristics of solid like deformation and liquid like flow in a fluid mixed system, failing to clarify a solid-fluid boundary of a yield stress fluid and not quantitatively considering an impact of shear duration on apparent viscosity of the yield stress fluid, being difficult to effectively characterize a rheological behavior and flow characteristics of a slope material during sliding under an action of complex waves, this embodiment provides a method for characterizing thixotropic characteristics of a submarine landslide mass material based on a rheological testing method, which clarifies the rheological behavior of an offshore silt seabed landslide mass/sediment during transporting and sliding by constructing a rheological model.

Measurement rheology is mainly a test method and means for researching rheological characteristics of a material, and is a basis for researching the rheological characteristics of the material. Parameters in a yield process are defined and tested from a perspective of phenomena, and the rheological behavior of the material may be displayed by obtaining original and detailed rheological data, so that this method becomes one of the powerful means to break through a bottleneck of complex rheological behavior researches.

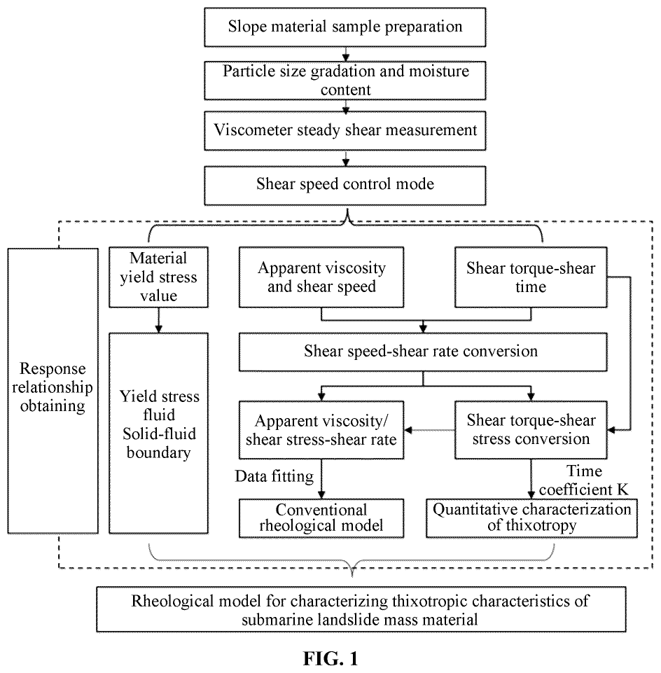

As shown in FIG. 1, the method specifically includes the following operations:

performing a steady shear on a landslide mass material by setting a shear speed and a shear time to perform a yield stress test, an apparent viscosity test, and a thixotropy test on the landslide mass material;
determining a solid-fluid transition boundary of the landslide mass material according to a varying law of a yield stress value with the shear time in the yield stress test;
performing conversions between a shear speed and a shear rate and between a shear torque and a shear stress according to varying laws of apparent viscosity-shear speed and shear torque-shear speed of the landslide mass material at different shear times in the apparent viscosity test to obtain varying laws of the apparent viscosity and the shear stress at different shear rates to construct a conventional rheological model of the landslide mass material;
constructing a thixotropy characterization equation of the landslide mass material according to the varying laws of the shear stress and the apparent viscosity with the shear time in the thixotropy test; and
constructing a rheological model for characterizing the thixotropic characteristics of the submarine landslide mass material according to the solid-fluid transition boundary, the conventional rheological model, and the thixotropy characterization equation to obtain steady-state rheological characteristics and flow behaviors of the submarine landslide mass material.

In this embodiment, a landslide mass material sample of seabed slope sediment is prepared by preparing sand-mud-water fluid mixtures with different material components, proportions, particle size features, and moisture contents, is uniformly stirred, and stands for later use.

Specifically, compositions of the landslide mass material of an offshore silt seabed are investigated and surveyed to determine main material components of the silt seabed and proportion variation ranges of the main material components. In view that the landslide mass material will present different flow behaviors and rheological characteristics under different particle size compositions, proportions, and moisture contents, so the landslide mass material sample is prepared by comprehensively considering particle size gradations and the moisture contents of the material components. Sample remodeling may alternatively be performed by using samples collected on site to finally obtain landslide mass material mixed fluid samples with different moisture contents.

In this embodiment, steady shear measurement is performed on the landslide mass material sample by using a rotary rheometer or a viscometer to obtain basic data for constructing the rheological model of the submarine landslide mass material. Specifically, different shear speeds and shear durations are set by using a shear speed control mode to perform the yield stress test, the apparent viscosity test, and the thixotropy test, and varying curves of a yield stress value, an apparent viscosity value, a shear torque value, and a shear torque value with the shear speed/shear time of the landslide mass material sample are recorded.

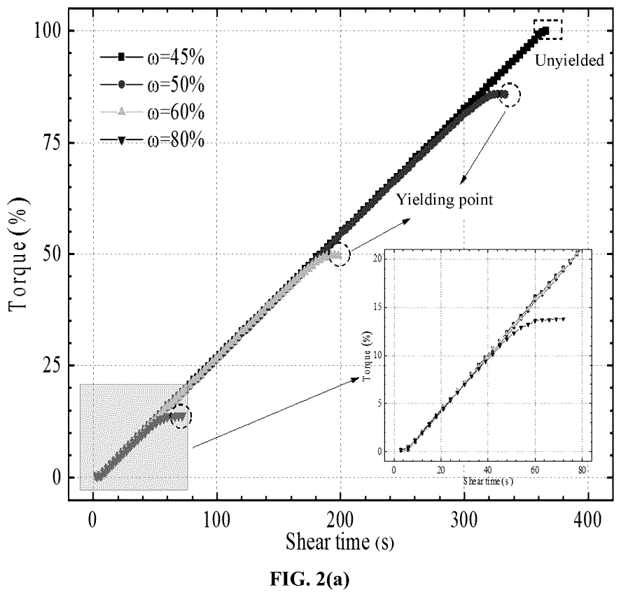

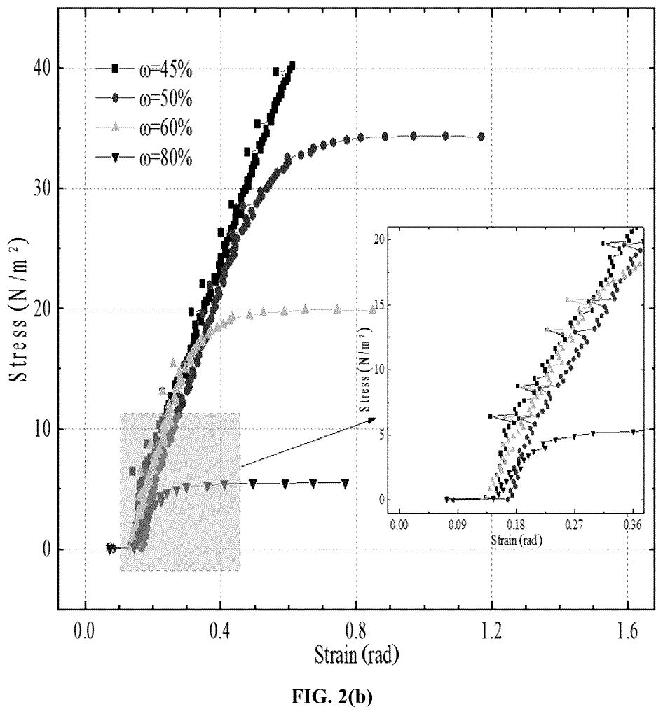

In this embodiment, as shown in FIG. 2(a) and FIG. 2(b), steady shear is performed on the landslide mass material sample by setting different constant shear speeds based on the prepared or remodeled landslide mass material sample, so as to perform the yield stress test, analyze yield strength characteristics of the landslide mass material sample, and determine the solid-fluid transition boundary of the landslide mass material according to a varying law of the yield stress value with the shear time.

Specifically, a constant shear speed is set to perform steady shear yield stress tests at different moisture contents to obtain a yield characteristic curve of the landslide mass material sample. After a certain shear duration, as the shear stress increases gradually, whether the landslide mass material sample tends to a steady shear stress value is determined according to a variation of the shear stress value of the landslide mass material sample with the shear time, and the yield stress value at this moment is a critical yield stress of the solid-fluid transition boundary after a material system yields.

In this embodiment, different shear speeds and shear durations are set in stages in the apparent viscosity test, and a conventional rheological model of the landslide mass material sample is fitted and constructed based on a measured apparent viscosity curve.

Specifically, conversions between a shear speed and a shear rate and between a shear torque and a shear stress are performed according to varying laws between an apparent viscosity and the shear speed and between the shear torque and the shear speed of the landslide mass material sample are performed at different shear times to obtain a relationship between the apparent viscosity/the shear stress and the shear rate, and the conventional rheological model of the landslide mass material is constructed through the varying laws of the apparent viscosity and the shear stress at different shear rates.

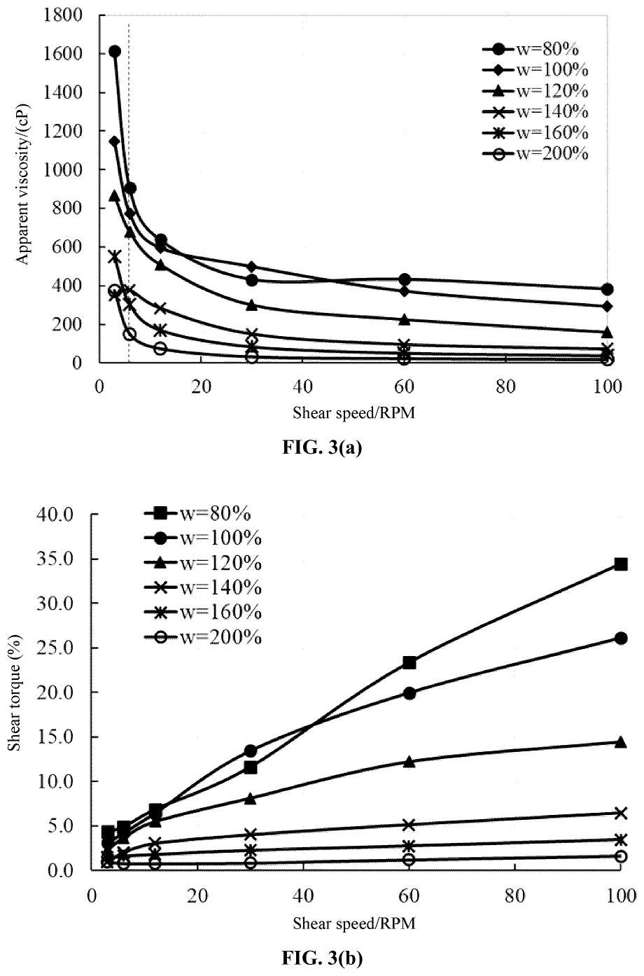

Further, different shear speeds are set to perform the apparent viscosity test of the landslide mass material sample to obtain conventional rheological curves of the apparent viscosity and the shear speed, and the shear torque and the shear speed shown in FIG. 3(a) and FIG. 3(b). Conversion relationships are respectively determined based on a test container, a test specification of the viscometer, and a rotor geometrical relationship to perform the conversions between the shear speed and the shear rate, and between the shear torque and the shear stress to obtain varying curves of the apparent viscosity and the shear stress under different shear rates, thereby fitting to obtain the conventional rheological model for characterizing the landslide mass material.

Further, the conventional rheological model includes an H-B rheological model, a Cross rheological model, a power law model, a Bingham model, and the like.

In this embodiment, the thixotropy test at a corresponding shear speed and shear time is performed based on different shear speeds and shear durations in the apparent viscosity test in the thixotropy test to obtain a varying relationship of the shear stress and the apparent viscosity with the shear time, meanwhile, a time dependent thixotropy characterization coefficient K is introduced to quantitatively characterize non-Newtonian fluid characteristics of the landslide mass material, that is, thixotropy characterization, and a thixotropy characterization equation is constructed.

Further, the thixotropy test at the corresponding shear time and shear speed refers to: the shear speed in the apparent viscosity test gradually increases to a maximum shear speed value. In the thixotropy test, a shear duration that is the same as that during the apparent viscosity test is maintained in the process that the shear speed increases to the maximum value first, and then the shear speed is decreased sequentially corresponding to a shear speed increasing process and the shear duration in the apparent viscosity test, but the corresponding shear duration is still maintained.

It may be understood that, the thixotropy test is essentially still the apparent viscosity test of the sample material, and physical quantities obtained by the thixotropy test are still interrelationships between the apparent viscosity/the shear torque—the shear speed/the shear time. The thixotropy test may be divided into two main parts. The first part is completely the same as an apparent viscosity test process. The second part is to reverse the test process of the first part (as introduced above). The physical quantities obtained by these two parts are the same as the physical quantities obtained in a conventional apparent viscosity test.

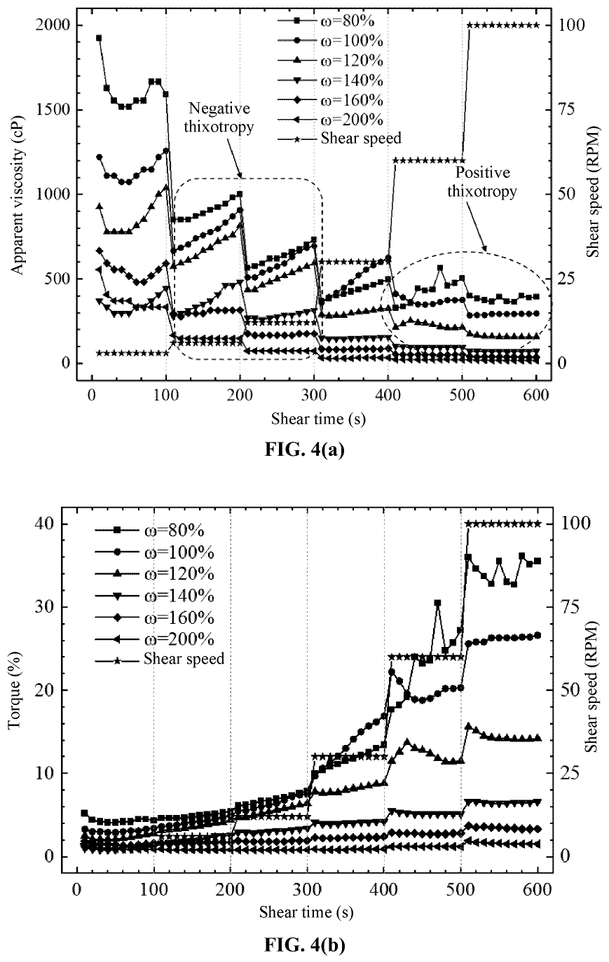

Further, an offshore submarine landslide mass material exhibits significant thixotropic flow characteristics. However, a landslide mass material system with thixotropy can only exhibit this property under an action of shearing. A constant shear speed is set to maintain continuous shear of the material system, and the landslide mass material will exhibit different rheological characteristics after undergoing a certain shear time, that is: thixotropic characteristics (positive thixotropy) and rheopectic characteristics (negative thixotropy) shown in FIG. 4(a) and FIG. 4(b), which are processes related to the shear time.

Further, the material system exhibits thixotropy because an equilibrium state between a formation rate and a destruction rate of an internal structure of the system is disrupted. Therefore, the thixotropy of the material system may be determined by comparing the formation and destruction rates.

It is worth noting that both formation and destruction of the structure require an action duration, so time and a final shear stress are main characteristics of the thixotropy, and are the characteristics exhibited by a thixotropic loop.

Further, in the characteristics exhibited by the thixotropic loop, the positive thixotropy is mathematically characterized as τᵤₚₛₜᵣₑₐₘ>τ_{downstream} at the same shear rate [symbol] , and the negative thixotropy is mathematically characterized as τᵤₚₛₜᵣₑₐₘ<τ_{downstream} at the same shear rate [symbol] .

That is, within a shear rate range, at any time, the material system with the thixotropy has a structure destructing and constructing processes at the same time, which are exhibited by the positive thixotropy and the negative thixotropy (the thixotropic loop). An upward curve in the thixotropic loop is determined by structural strength (the destruction rate), and a downward curve is determined by the formation rate.

In conclusion, a comprehensive relationship between time and a shear rate are to be considered at the same time when accurately describe the thixotropy of the material system. Therefore, the time dependent thixotropy characterization coefficient K is introduced in this embodiment to quantitatively characterize the magnitude of the thixotropy of the material system.

In one aspect, the time dependent thixotropy characterization coefficient K may characterize a varying law of an internal structure system with the shear time in a shear history, namely, a time varying law of a time-varying law of the apparent viscosity. In another aspect, the time dependent thixotropy characterization coefficient K may reflect a varying degree of an internal structure of the material caused by a variation of the shear rate, that is, the varying law of the apparent viscosity with the shear rate, which is also a conventional rheological model.

Therefore, the thixotropy characterization coefficient K of the material system at times t₁ to t₂ are obtained, using a rotational viscometer, by measuring system apparent viscosities η₁ and η₂ at the times t₁ and t₂ and measuring corresponding shear rates [symbol] and [symbol] .

$$K = \frac{{η}_{1} - {η}_{2}}{In ( {t}_{2} - {t}_{1} ) In ( {\dot{γ}}_{2} - {\dot{γ}}_{1} )} = \frac{{τ}_{1} \text{?}}{\text{?}} \qquad ( 1 )$$

$$\text{?} \text{indicates text missing or illegible when filed}$$

Complex measurement processes are accompanied by multiple stages of variations of the shear rates, so the thixotropy characterization coefficient K is constructed by an interval averaging method:

$$K = \frac{1}{n} {\sum }_{i = 1}^{n} ( \frac{{η}_{1} - {η}_{i}}{In ( {t}_{i} - {t}_{1} ) In ( {\dot{γ}}_{i} - {\dot{γ}}_{1} )} ) = \frac{1}{n} {\sum }_{i = 1}^{n} ( \frac{{τ}_{1} \text{?}}{\text{?}} ) \qquad ( 2 )$$

$$\text{?} \text{indicates text missing or illegible when filed}$$

wherein, i is an i^{th} duration of the shear rate, and n is an entire shear duration.

In real life, fluids may be classified into Newtonian fluids and non-Newtonian fluids. For example, common water belongs to the Newtonian fluids. When such fluids are subjected to a shear action, there is a linear relationship (viscosity) between a shear stress and a shear strain rate, and the viscosity does not vary with an increase of shear duration. The other type of fluids is the non-Newtonian fluids. There is a non-linear relationship (apparent viscosity) between a shear stress and a shear strain rate of the non-Newtonian fluids. Most fluids in production and daily life belong to the non-Newtonian fluids.

The non-Newtonian fluids may further be classified into non-time-varying non-Newtonian fluids and time-varying non-Newtonian fluids according to whether an apparent viscosity function of the non-Newtonian fluids is related to shear duration in a shear process. Specifically, for a second type of time-varying non-Newtonian fluids, the viscosity functions (the non-linear relationship between the shear stress and the shear strain rate) of such fluids are dependent of not only the shear strain rate, but also the shear duration. Thixotropic fluids are exactly one type of the time-varying non-Newtonian fluids.

The thixotropic fluids may further be classified into two types: positive thixotropic fluids (thixotropic fluids) and negative thixotropic fluids (rheopectic fluids). Conventionally, at a certain shear strain rate, a viscosity function of a positive thixotropic fluid decreases with time; and an apparent viscosity function of a negative thixotropic fluid increases with time.

The apparent viscosity of a non-time-varying non-Newtonian fluid is only a function of the shear strain rate, and is independent of time. In other words, an internal structure of the fluid is instantaneously adjusted after the shear strain rate varies. The shear stress and the apparent viscosity corresponding to the shear strain rate may be immediately obtained after varying the shear rate, and a structure adjustment time is short, so it is difficult to sensitively capture or reflect such suddenly varying time by an existing measurement technology. This is the meaning of non-time-varying. However, a structure is adjusted slowly for the time-varying non-Newtonian fluid (the thixotropic fluid).

Mechanical properties of the fluids are impacted by variations of system structures. Therefore, rheological characteristics of the fluids vary with time in a period of time of fluid structure adjustment until a new equilibrium structure is formed. A structure in this fluid system is constantly formed and is constantly destructed at the same time. The equilibrium structure refers to that a destruction rate and a formation speed of the structure are the same, and the system is in a dynamic equilibrium state. If a shear action stops, the structure of the fluid system restores to an original state, and this process is reversible. In short, the thixotropy can actually be regarded as a measure of resistance of the internal structure of the non-Newtonian fluid to adjustment.

However, at the present stage, researches on constitutive equations for thixotropic fluids are still far from meeting a requirement for effectively characterizing thixotropic characteristics of the thixotropic fluids. In many cases, empirical determination can only be performed through rheological tests, and operating processes are different.

In conclusion, to conveniently and efficiently measure the thixotropy, this embodiment provides the thixotropy characterization coefficient K.

In this embodiment, to accurately characterize non-Newtonian fluid characteristics of an offshore submarine landslide mass material and comprehensively consider yield characteristics of the material, a conventional rheological model, and time dependence of a rheological behavior, a rheological model for characterizing thixotropic characteristics of the submarine landslide mass material is constructed according to a solid-fluid transition boundary, the conventional rheological model, and a thixotropy characterization equation to obtain steady-state rheological characteristics and flow behaviors of the submarine landslide mass material, which provides effective theoretical guidance for researching transportation and accumulation of a landslide mass during a submarine landslide disaster.

This embodiment elaborates in detail a process for constructing a rheological model for characterizing thixotropic characteristics of the submarine landslide mass material and system equation parameter determination and correction methods involved. The above description is only intended to help understand the methods and core ideas of this embodiment. Meanwhile, for those of ordinary skill in the art, there will be variations in specific embodiments and application scopes according to the idea of the present disclosure. In conclusion, the above content is not to be construed as a limitation to this embodiment.

### Embodiment 2

This embodiment provides a system for characterizing thixotropic characteristics of a submarine landslide mass material, including:
a test module, configured to perform steady shear on a landslide mass material by setting a shear speed and a shear time to perform a yield stress test, an apparent viscosity test, and a thixotropy test on the landslide mass material;
a first response relationship obtaining module, configured to determine a solid-fluid transition boundary of the landslide mass material according to a varying law of a yield stress value with the shear time in the yield stress test;
a second response relationship obtaining module, configured to perform conversions between a shear speed and a shear rate and between a shear torque and a shear stress according to varying laws of apparent viscosity-shear speed and shear torque-shear speed of the landslide mass material at different shear times in the apparent viscosity test to obtain varying laws of the apparent viscosity and the shear stress at different shear rates to construct a conventional rheological model of the landslide mass material;
a third response relationship obtaining module, configured to construct a thixotropy characterization equation of the landslide mass material according to the varying laws of the shear stress and the apparent viscosity with the shear time in the thixotropy test; and
a thixotropic characteristic characterization module, configured to construct a rheological model for characterizing the thixotropic characteristics of the submarine landslide mass material according to the solid-fluid transition boundary, the conventional rheological model, and the thixotropy characterization equation to obtain steady-state rheological characteristics and flow behaviors of the submarine landslide mass material.

Here, it is to be noted that, the above modules correspond to the operations described in Embodiment 1. The above modules and the corresponding operations implemented in the same examples and application scenarios, but are not limited to the content disclosed in Embodiment 1. It is to be noted that, the above modules, as part of the system, may be executed in a computer system including, for example, a set of computer-executable instructions.

In more embodiments, further provided is:

An electronic device, including a memory, a processor, and computer instructions stored in the memory and run in the processor. When the processor runs the computer instructions, implementing the method in Embodiment 1. Repetition is omitted here for simplicity.

It is to be understood that, in this embodiment, the processor may be a central processing unit (CPU). The processor may alternatively be other general-purpose processors, digital signal processors (DSPs), application-specific integrated circuits (ASICs), field programmable gate arrays (FPGAs) or other programmable logic devices, discrete gates, or transistor logic devices, or discrete hardware components, or the like. The general-purpose processor may be a microprocessor, or the processor may be any conventional processor or the like.

The memory may include a read-only memory and a random access memory, and provide instructions and data for the processor. A part of the memory may alternatively include a non-volatile random access memory. For example, the memory may further store device type information.

A non-transitory computer-readable storage medium is configured to store computer instructions. The method in Embodiment 1 is completed when the computer instructions are executed by a processor.

The method in Embodiment 1 may be directly performed and completed by using a hardware processor, or may be performed and completed by using a combination of hardware and software modules in the processor. The software module may be located in a storage medium that is mature in the art, such as a random access memory (RAM), a flash memory, a read-only memory (ROM), a programmable ROM, an electrically erasable programmable memory, or a register. The storage medium is located in the memory. The processor reads information in the memory and completes the steps of the methods in combination with hardware thereof. To avoid repetition, details are not described in detail herein.

Those of ordinary skill in the art may be aware that units, namely, algorithm operations, in each example described with reference to this embodiment can be implemented by electronic hardware or a combination of computer software and electronic hardware. Whether the functions are executed in a mode of hardware or software depends on particular applications and design constraint conditions of the technical solutions. Those skilled in the art may use different methods to implement the described functions for each particular application, but it is not to be considered that this implementation goes beyond the scope of this application.

Specific embodiments of the present disclosure are described above with reference to the accompanying drawings, but are not intended to limit the scope of protection of the present disclosure. Those skilled in the art are to be understood that various modifications or deformations made without creative efforts based on the technical solutions of the present disclosure still fall within the scope of protection of the present disclosure.

1. A method for characterizing thixotropic characteristics of a submarine landslide mass material, comprising:
performing steady shear on a landslide mass material by setting a shear speed and a shear time to perform a yield stress test, an apparent viscosity test, and a thixotropy test on the landslide mass material;
determining a solid-fluid transition boundary of the landslide mass material according to a varying law of a yield stress value with the shear time in the yield stress test;
performing conversions between a shear speed and a shear rate and between a shear torque and a shear stress according to varying laws of apparent viscosity-shear speed and shear torque-shear speed of the landslide mass material at different shear times in the apparent viscosity test to obtain varying laws of the apparent viscosity and the shear stress at different shear rates to construct a conventional rheological model of the landslide mass material;
constructing a thixotropy characterization equation of the landslide mass material according to the varying laws of the shear stress and the apparent viscosity with the shear time in the thixotropy test; and
constructing a rheological model for characterizing the thixotropic characteristics of the submarine landslide mass material according to the solid-fluid transition boundary, the conventional rheological model, and the thixotropy characterization equation to obtain steady-state rheological characteristics and flow behaviors of the submarine landslide mass material.

2. The method for characterizing thixotropic characteristics of a submarine landslide mass material according to claim 1, wherein in the yield stress test, a constant shear speed is set to perform steady shear yield stress tests at different moisture contents to obtain a yield characteristic curve of the landslide mass material; and after a certain shear duration, whether the landslide mass material tends to a steady shear stress value is determined according to a variation of the shear stress value of the landslide mass material with the shear time, and the yield stress value at this moment is a critical yield stress of the solid-fluid transition boundary after the landslide mass material yields.

3. The method for characterizing thixotropic characteristics of a submarine landslide mass material according to claim 1, wherein in the apparent viscosity test in the thixotropy test, the thixotropy test at a corresponding shear speed and shear time is performed based on different shear speeds and shear times to obtain variations of the shear stress and the apparent viscosity with the shear time.

4. The method for characterizing thixotropic characteristics of a submarine landslide mass material according to claim 3, wherein the thixotropy test at the corresponding shear speed and shear time is as follows: the shear speed in the apparent viscosity test gradually increases to a maximum shear speed, the same shear time is maintained in a process that the shear speed increases to a maximum value in the thixotropy test, the shear speed is decreased sequentially corresponding to a shear speed increasing process and the corresponding shear time in the apparent viscosity test, and the corresponding shear time is maintained.

5. The method for characterizing thixotropic characteristics of a submarine landslide mass material according to claim 1, wherein a time dependent thixotropy characterization coefficient is introduced in the thixotropy test to quantitatively characterize non-Newtonian fluid characteristics of the landslide mass material.

6. The method for characterizing thixotropic characteristics of a submarine landslide mass material according to claim 5, wherein the time dependent thixotropy characterization coefficient K is as follows:
$$K = \frac{1}{n} {\sum }_{i = 1}^{n} ( \frac{{η}_{1} - {η}_{i}}{In ( {t}_{i} - {t}_{1} ) In ( {\dot{γ}}_{i} - {\dot{γ}}_{1} )} )$$
wherein, i is an i^{th} duration of the shear rate, n is an entire shear duration, \dot{γ}₁ and \dot{γ}ᵢ are the shear rates corresponding to time t₁ and time tᵢ, and η₁ and ηᵢ are apparent viscosities of the system corresponding to time t₁ and time tᵢ.

7. The method for characterizing thixotropic characteristics of a submarine landslide mass material according to claim 1, wherein a landslide mass material sample is prepared by comprehensively considering particle size gradation and moisture content of landslide mass material components; or, sample remodeling is performed by using samples collected on site to obtain landslide mass material samples with different moisture contents.

8. A system for characterizing thixotropic characteristics of a submarine landslide mass material, comprising:
a test module, configured to perform steady shear on a landslide mass material by setting a shear speed and a shear time to perform a yield stress test, an apparent viscosity test, and a thixotropy test on the landslide mass material;
a first response relationship obtaining module, configured to determine a solid-fluid transition boundary of the landslide mass material according to a varying law of a yield stress value with the shear time in the yield stress test;
a second response relationship obtaining module, configured to perform conversions between a shear speed and a shear rate and between a shear torque and a shear stress according to varying laws of apparent viscosity-shear speed and shear torque-shear speed of the landslide mass material at different shear times in the apparent viscosity test to obtain varying laws of the apparent viscosity and the shear stress at different shear rates to construct a conventional rheological model of the landslide mass material;
a third response relationship obtaining module, configured to construct a thixotropy characterization equation of the landslide mass material according to the varying laws of the shear stress and the apparent viscosity with the shear time in the thixotropy test; and
a thixotropic characteristic characterization module, configured to construct a rheological model for characterizing the thixotropic characteristics of the submarine landslide mass material according to the solid-fluid transition boundary, the conventional rheological model, and the thixotropy characterization equation to obtain steady-state rheological characteristics and flow behaviors of the submarine landslide mass material.

9. An electronic device, comprising a memory, a processor, and computer instructions stored in the memory and running in the processor, and the method according to claim 1 being completed when the computer instructions are run by the processor.

10. A non-transitory computer-readable storage medium, being configured to store computer instructions, and the method according to claim 1 being completed when the computer instructions are executed by a processor.
